(19) United States
(12) Patent Application Publication
(10) Pub. No.: US 2010/0184117 A1
(43) Pub. Date: Jul. 22, 2010

(54) METHOD AND SYSTEM FOR CHARACTERIZING A PIGMENTED BIOLOGICAL TISSUE

(75) Inventors: Zoran Cerovic, Paris (FR); Nicolae Moise, Gif Sur Yvette (FR); Gwendal Latouche, Paris (FR); Yves Goulas, Le Plessis Robinson (FR)

Correspondence Address: YOUNG & THOMPSON, 209 Madison Street, Suite 500, Alexandria, VA, 22314, US

(73) Assignees: FORCE-A, ORSAY (FR); CENTRE NATIONAL DE LA RECHERCHE SCIENTIFIQUE, PARIS (FR); UNIVERSITE PARIS SUD, ORSAY (FR)

(21) Appl. No.: 12/602,530

(22) PCT Filed: May 27, 2008
(86) PCT No.: PCT/FR2008/050911

§ 371 Date: Dec. 1, 2009

(30) Foreign Application Priority Data
Jun. 1, 2007 (FR) 0703924

(51) Int. Cl. C12Q 1/02

(52) U.S. Cl. 435/29

(57) ABSTRACT

Method for determining the content of a non-fluorescent chromophorous first compound, in a biological tissue including a fluorescent chromophorous second compound, the method includes at least one iteration of the following operations:
emission, in the direction of said tissue, of a first reference optical radiation, and a second measurement optical radiation, each chosen so as to induce a fluorescence radiation of the second compound, each of the first and second radiations being partially absorbed by the first compound,
measuring the fluorescence radiations induced by each of the first and second radiations, and
determining, from the measurement, of the content of the first compound in the tissue, wherein the method includes at least one compensation of a measurement saturation due to too high an absorption of the measurement radiation by the first compound, the compensation comprising a choice, for the measurement radiation, of a wavelength corresponding to a lower absorption in the absorption spectrum of the first compound.

The present invention relates to a method for characterizing a pigmented biological tissue. It also relates to a system implementing this method.

More particularly, the invention relates to a method and a system for characterizing a tissue of a biological entity, such as a berry, comprising a first chromophorous compound which is only slightly fluorescent or not fluorescent, such as an anthocyanin or a flavonol, and a second fluorescent chromophorous compound such as chlorophyll. The characterization aims to determine the content of the first compound in a biological tissue. Such a characterization of a biological tissue is highly significant. For example in the field of wine production, the anthocyanin content of the grapes represents information concerning their maturity and the quality of the wine obtained from these grapes. In the nutritional field, the flavonol content of the skin of fruits and vegetables is an indicator of their nutritional value.

The document FR 2 830 325 discloses an optical instrument which makes it possible to measure, by a screening effect, light absorption characteristics of a sample of biological tissue comprising a first non-fluorescent chromophorous compound and a second fluorescent chromophorous compound. The method disclosed in this document involves measuring the fluorescence induced by excitation of the second compound by a first and a second radiation of different wavelengths illuminating the biological tissue sample. One of these so-called reference radiations is slightly or not at all absorbed by the first compound whereas the other, so-called measurement radiation is relatively well absorbed. By measuring the fluorescence induced by the two radiations and finding the ratio of the measured fluorescences, the method makes it possible to measure the light absorption characteristics of the sample and as a result the content of the first compound in the biological tissue.

However, the development of the content of the first, non-fluorescent, chromophorous compound causes a saturation of the measurement of the fluorescence radiation induced by the first radiation. When the concentration of the first compound in the tissue increases, the absorption of the measurement radiation by the first compound also increases. The fluorescence radiation induced by the measurement radiation becomes increasingly weak and can no longer be distinguished from the measurement noise. It may also be the case that, when the concentration of the first compound in the tissue increases, the reference radiation begins to be affected. In the present description, these effects will be called “saturation”.

Most of the methods and systems currently known are limited to characterizing a biological tissue until saturation is reached. These systems include those based on colorimetry, which as well as saturation, encounter problems linked with the deposits that can be present on the biological tissue and can affect the measurements. Systems based on infrared spectroscopy can also be mentioned, which encounter problems linked with the presence of water in the biological tissue and which carry out a determination of the content in the first compound based on an empirical chemometric deduction.

Other methods and systems for characterizing a biological tissue allow saturation to be avoided by dilution operations in the laboratory. A drawback of such methods and systems is that they are destructive.

A purpose of the invention is to propose a novel method and system allowing a biological tissue to be characterized beyond saturation in a non-destructive, non-invasive fashion.

Another purpose of the invention is to propose a new method and a system allowing a biological tissue to be characterized beyond saturation and which can be used in situ.

The invention thus proposes a method for determining the content of a non-fluorescent chromophorous compound, called the first compound, of a biological tissue of a biological entity, the biological tissue comprising moreover a fluorescent chromophorous compound, called the second compound, this method comprising at least one iteration of the following operations:
emission by emission means, in the direction of the tissue, of a first so-called measurement optical radiation, and a second so-called reference optical radiation, each chosen so as to induce a fluorescence radiation of the second compound, each of the first and second radiations being partially absorbed by the first compound,
measuring, by measurement means, fluorescence radiations induced by the first and second radiations, and
determining, from the measurement, the content of the first compound in the tissue.

The method according to the invention is characterized in that it comprises moreover at least one compensation of a measurement saturation due to too high an absorption of the measurement radiation by the first compound, due to too high a content of the first compound, the compensation comprising a choice, for the measurement radiation, of a wavelength corresponding to a lower absorption in the absorption spectrum of the first compound.

The method according to the invention thus makes it possible to carry out the characterization of a biological tissue of a biological entity, beyond a saturation due to too high a content of the first compound, i.e., too high an absorption of the measurement radiation by the first compound, by shifting the wavelength of the measurement radiation towards a lower-absorption wavelength in the absorption spectrum of the first compound. Moreover, since it does not require operations in the laboratory, the method according to the invention is non-destructive, non-invasive and can be implemented in situ.

Advantageously, the method according to the invention can moreover comprise an emission, by the emission means, in the direction of the tissue, of a third so-called additional optical radiation, chosen so as to induce a fluorescence radiation of the second compound. In this case, the method according to the invention comprises measuring the fluorescence radiation induced by the additional radiation. This measurement is then used for determining whether the reference radiation is affected or not by the first compound. In fact, according to the ratio of the fluorescence radiation induced by the two radiations, reference and additional, and the variation of this ratio, it is possible to determine whether or not the reference radiation is affected by the first compound. When it is detected that the reference radiation is affected by the first compound, a saturation compensation must be carried out, for example by inverting the measured ratio.

According to, a first aspect of the invention, when the content of the tissue in the first compound increases such that the first compound affects the reference radiation, the wavelength chosen for the measurement radiation after saturation corresponds to a wavelength situated around the wavelength of the reference radiation before saturation. According to this first aspect of the invention, when saturation occurs, a new wavelength is chosen for the measurement radiation. The new wavelength chosen for the measurement radiation can be the wavelength of the reference radiation before saturation. When the distance between the biological tissue and the emission means and the measurement means is fixed, it is not necessary to use a reference radiation. However, the first measurement radiation, even when saturated, can be used in the measured ratio.

When the distance between the biological tissue and the emission means and the measurement means is not fixed, the wavelength chosen for the reference radiation after saturation can correspond to a wavelength which is only a little, or not at all, affected by the content of first compound. More particularly, the wavelength of the reference radiation after the saturation can correspond to the wavelength of the additional radiation.

According to a second aspect of the invention, when the reference wavelength is not affected by the content of the first compound in the tissue, the wavelength chosen for the measurement radiation corresponds to a lower absorption by the first compound than a so-called limit wavelength, for which a potential saturation would be produced for an expected maximum content for the first compound in the biological tissue. During a measurement series, the potential maximum content of the first compound can be determined. According to the potential maximum value and the absorption spectrum of the first compound, it is possible to calculate a maximum absorption that can potentially be obtained. A so-called limit wavelength, corresponding to a saturation for maximum absorption, can be determined for the measurement radiation. Thus, if a wavelength corresponding to a lower absorption than the limit wavelength is chosen for the measurement radiation, no saturation with respect to the measurement radiation will be produced during the measurement series. The limit wavelength can be determined as a function of an absorption peak. In this case, the measurement wavelength is chosen with respect to the absorption peak. The method according to the invention can then comprise a determination of the shift with respect to the absorption peak so as to avoid saturation or to obtain values for the content of the first compound developing in a more linear fashion.

According to a third aspect of the invention, when the reference wavelength is not affected by the content of the first compound in the tissue, the saturation compensation can be carried out when the content of the first compound in the tissue causes a measurement saturation, said compensation being reiterated at each saturation. When, after a measurement series carried out with a measurement radiation having a first wavelength corresponding for example to an absorption peak, a first saturation is achieved, the wavelength of the measurement radiation can be shifted towards a second wavelength corresponding to an absorption lower than the first wavelength such that this second wavelength does not cause measurement saturation. Then a second measurement series can be conducted, until a new saturation is reached. The wavelength of the measurement radiation can then be shifted towards a third wavelength corresponding to a lower absorption than the second wavelength, and so on. On choosing a new wavelength, the difference in absorption can be taken into account in calculating the content of the first compound in the tissue. This difference in absorption can be obtained by studying the absorption spectrum of the first compound providing calibration values for the different wavelengths chosen.

The three aspects of the invention described above can advantageously be combined.

Advantageously, the method according to the invention can comprise a modification of the wavelength of the measurement radiation and/or the reference radiation as a function of a physico-chemical characteristic having an influence on the absorption spectrum of the first compound and/or on the fluorescence spectrum of the second compound.

The physico-chemical characteristic can be a characteristic chosen from the following list:
the pH of the biological tissue
the effects of co-pigmentation affecting the first compound, and
the effects of the changes in the support structures of the second compound.

The wavelength of the measurement radiation and/or the reference radiation can also be modified according to an appearance in the biological tissue of a new compound, for example by covalent change, modifying the absorption spectrum of the first compound or the fluorescence spectrum of the second compound.

Advantageously, the measurement radiation (or the reference radiation) can be emitted at a predetermined intensity and the reference radiation (or the measurement radiation) at a variable intensity so that the fluorescence radiation induced by each of said reference and measurement radiations are of equal intensity.

The intensity of the first radiation can be adjusted according to a so-called control signal, with respect to the induced fluorescence radiations, on the one hand by the measurement radiation, and on the other hand by the reference radiation.

Moreover, the intensities of the measurement and reference radiations can vary alternately, by phase shifting, at a predetermined frequency which can be 1 kHz, for example.

The method according to the invention can advantageously comprise a synchronization by phase modulation of the radiations emitted by the emission means.

Each of the measurement radiations can be emitted in the form of pulses.

The determination of the content of the first compound can comprise a calculation of the ratio (fluorescence induced by the reference radiation) divided by (fluorescence induced by the measurement radiation).

According to an advantageous feature of the method according to the invention, the measurement of the fluorescence radiations can be carried out:
either on a tissue sample taken from the biological entity;
or directly on the biological entity bearing the tissue, in a non-destructive and non-invasive fashion, i.e. without having to take a sample.

The method according to the invention can advantageously comprise a determination of the development over time of the content of the first compound in the biological tissue. This development can be determined according to a unit of time which can be the day. It can, for example be expressed as a graph, showing the development of the content of compound as a function of the unit of time chosen.

In a non-limitative embodiment of the first aspect of the method according to the invention, the biological tissue is the skin of a grape berry, the first compound is an anthocyanin and the second compound is chlorophyll. When the distance between the tissue and the emission means and the measurement means is fixed:
the wavelength of the measurement radiation before saturation is 530 nm;
the wavelength of the reference radiation before saturation is of 650 nm;
the wavelength of the additional radiation is 450 nm;
the wavelength of the measurement radiation after saturation is 650 nm;
and there is no need for a reference radiation after saturation.
   Still in the case of this example, when the distance between the tissue and the emission means and the measurement means is not fixed:
the wavelength of the measurement radiation before saturation is 530 nm;
the wavelength of the reference radiation before saturation is 650 nm;
the wavelength of the additional radiation is 450 nm;
the wavelength of the measurement radiation after saturation is 650 nm;
the wavelength of the reference radiation after saturation is 450 nm;
   In this case, the anthocyanin content of the skin of a grape berry can be determined according to the following relationships:

ANTH_{before saturation}=logFER_ANTH_GREEN−logFER_CHL_{G}

ANTH_{after saturation}=C+logFER_ANTH_RED−logFER_CHL_{R}

with,
1. logFER_ANTH_GREEN=log(FRFred/FRFgreen)
2. logFER_ANTH_RED=log(FRFblue/FRFred) and
3. logFER_CHL_{G}=log(FRFred/FRFgreen)
4. logFER_CHL_{R}=log(FRFblue/FRFred)
   In these expressions:
FRFred corresponds to the fluorescence radiation of the chlorophyll induced by a radiation emitted at 650 nm, i.e. the reference radiation before saturation and the measurement radiation after saturation;
FRFgreen corresponds to the fluorescence radiation of the chlorophyll induced by a radiation emitted at 530 nm, i.e. the measurement radiation before saturation;
FRFblue corresponds to the fluorescence radiation of the chlorophyll induced by a radiation emitted at 450 nm, i.e. the additional radiation before saturation and the reference radiation after saturation;
The expressions 3 and 4 correspond to at least one measurement carried out before the appearance of anthocyanins in the skin of the grape berry. It serves as a reference for the measurements carried out after the appearance of the anthocyanins; and
C is a constant such that ANTH_{after saturation} is equal to ANTH_{before saturation} at the time of saturation.

In another non-(imitative example and according to the third aspect of the invention, the biological tissue is the skin of a grape berry, the first compound is a flavonol, the second compound is chlorophyll:
the wavelength of the measurement radiation before saturation is approximately 375 nm;
the wavelength of the reference radiation before saturation is approximately 650 nm;
the wavelength of the measurement radiation after saturation is approximately 450 nm; and
the wavelength of the reference radiation after saturation remains approximately 650 nm;

According yet another aspect of the invention a system is proposed implementing the method according to the invention.

Other characteristics and advantages of the invention will become apparent on examination of the detailed description of an embodiment which is in no way limitative, and the attached drawings in which:

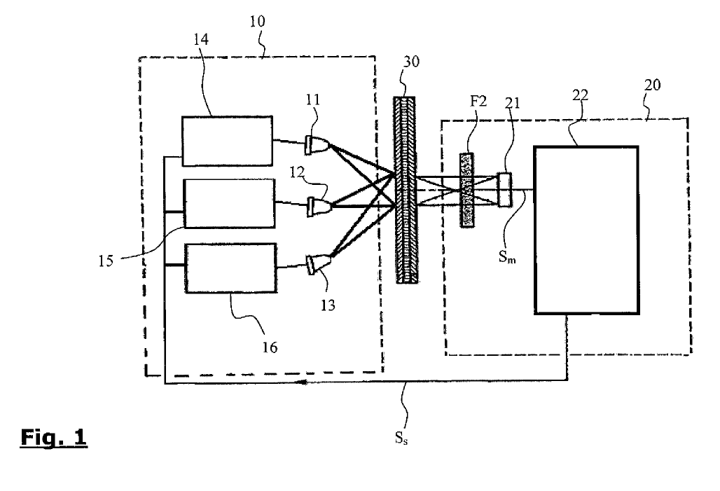

FIG. 1 is a diagrammatic representation of a first embodiment of the system according to the invention;

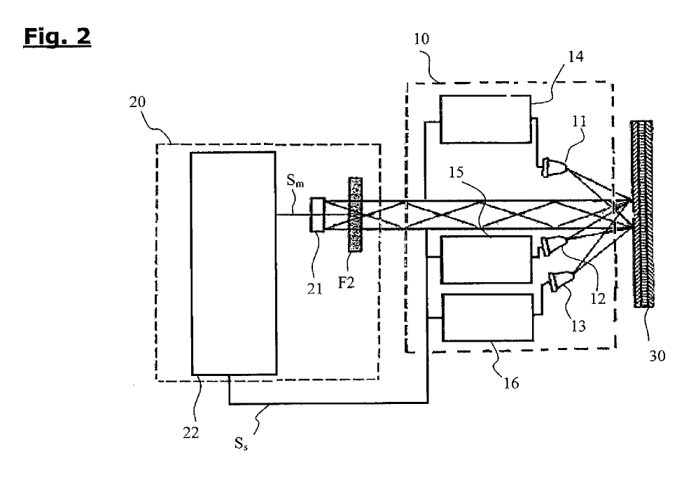

FIG. 2 is a diagrammatic representation of a second embodiment of the system according to the invention;

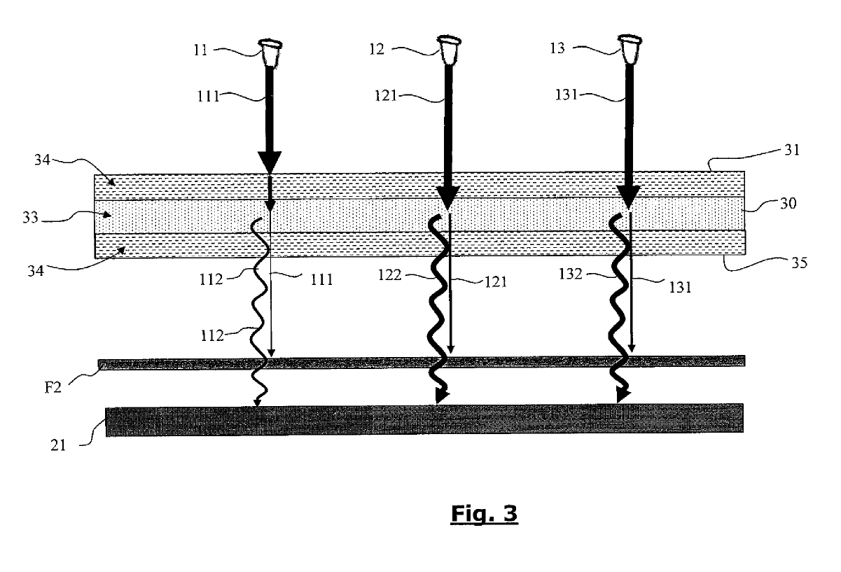

FIG. 3 is a diagrammatic representation of the measurement principle, in the first embodiment of the system according to the invention;

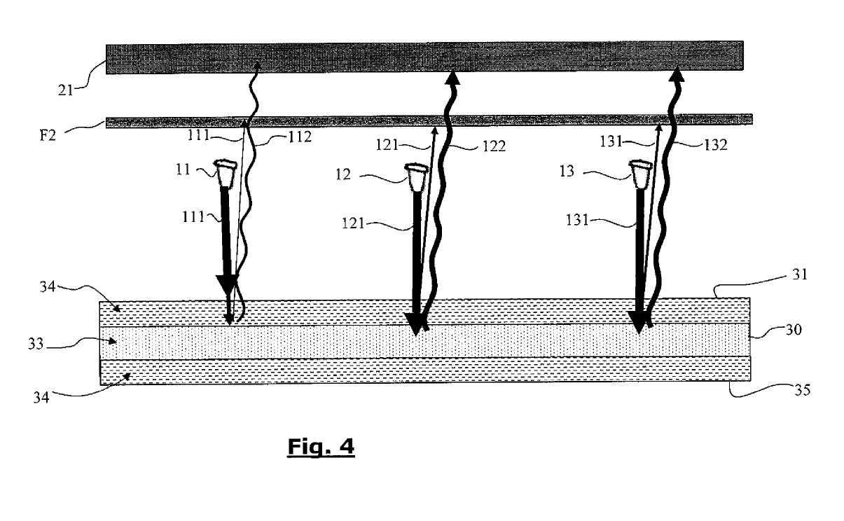

FIG. 4 is a diagrammatic representation of the measurement principle, in the second embodiment of the system according to the invention;

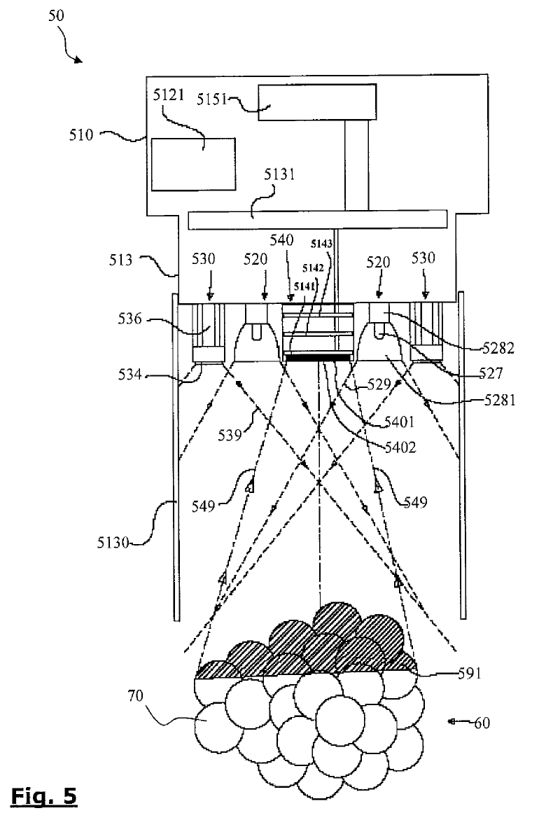

FIGS. 5, 6 and 7 are a diagrammatic representation of a system according to the second embodiment of the invention;

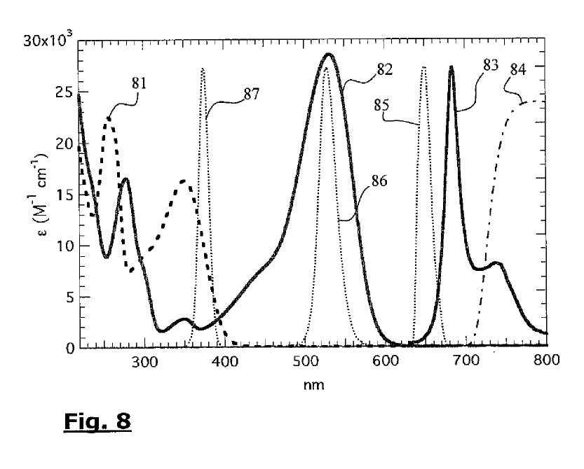

FIG. 8 is a representation of the spectra of fluorescence of chlorophyll, absorption of an anthocyanin, absorption of a flavonol as well as different radiations and filters used according to the first aspect of the method according to the invention;

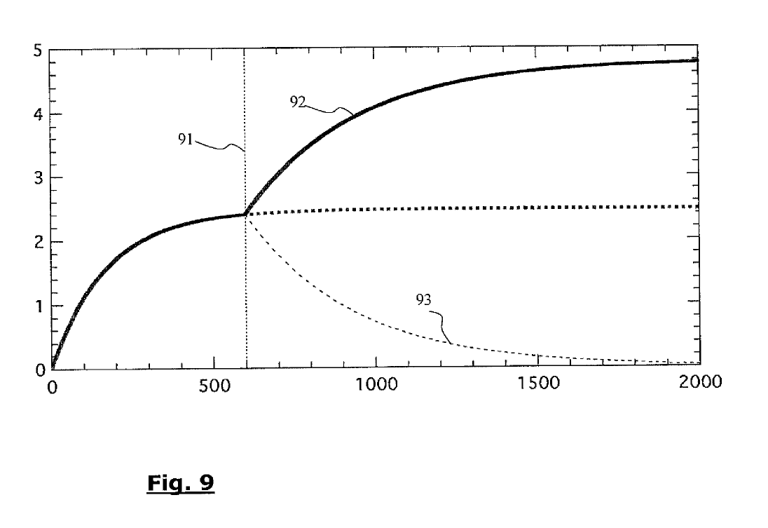

FIG. 9 is a representation of the results obtained during the measurement of the anthocyanin content of the skin of a grape berry according to the first aspect of the method according to the invention;

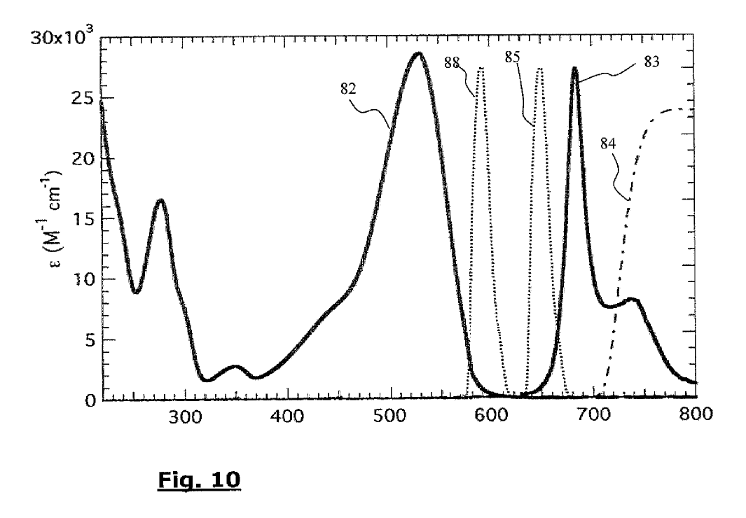

FIG. 10 is a representation of the spectra of fluorescence of chlorophyll, absorption of an anthocyanin, as well as of the different radiations and filters used according to the second aspect of the method according to the invention;

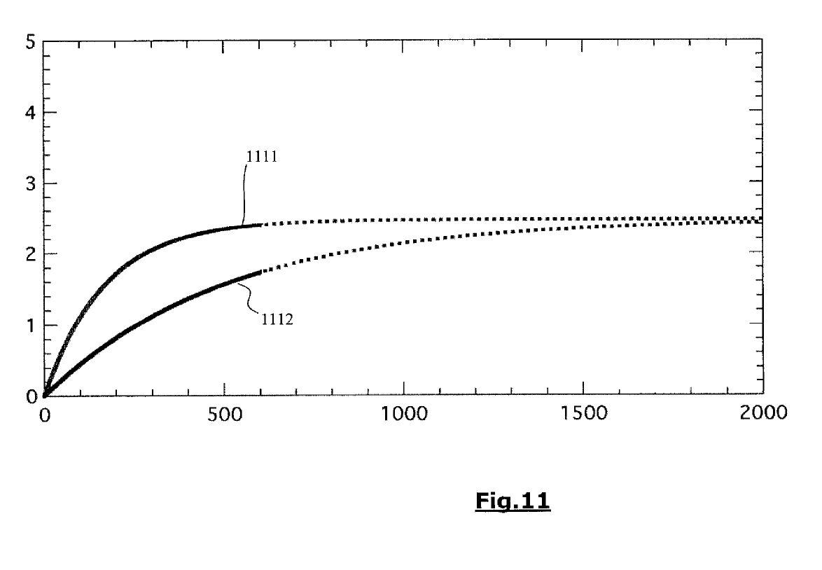

FIG. 11 is a representation of the results obtained during the measurement of the anthocyanin content of the skin of a grape berry according to the second aspect of the method according to the invention;

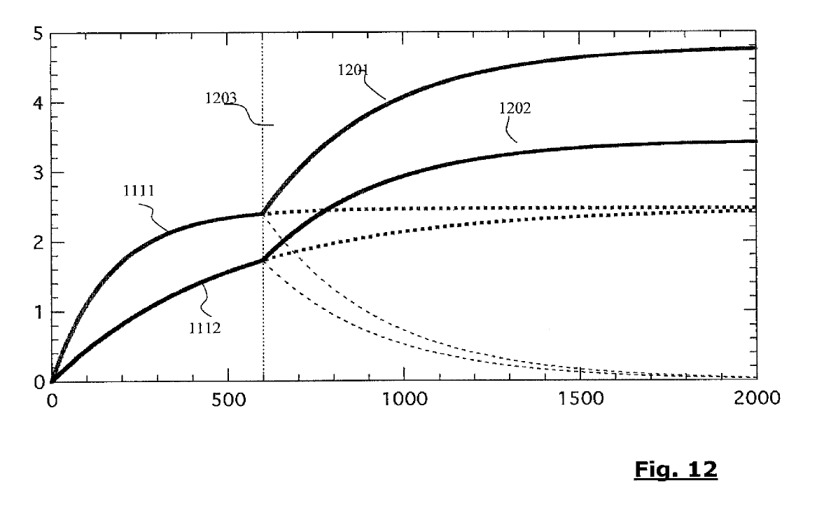

FIG. 12 is a representation of the results obtained during the measurement of the anthocyanin content of the skin of a grape berry according to a combination of the first and second aspects of the method according to the invention;

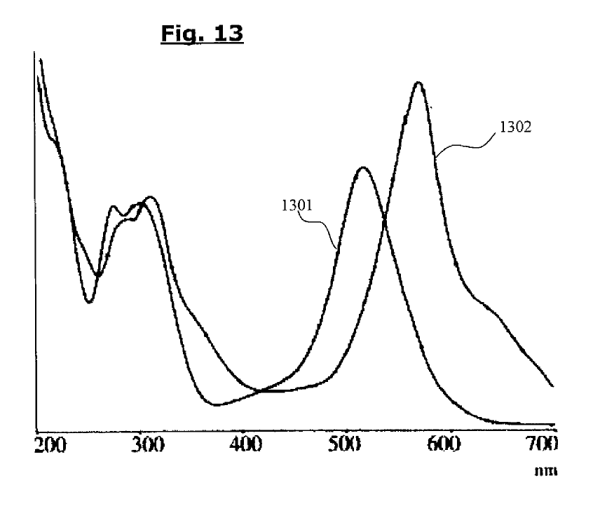

FIG. 13 is a representation of an example of the variation of the absorption spectrum of anthocyanins as a function of the pH.

The non-limitative embodiment that will be described below relates to a system for the measurement of the content of a first non-fluorescent chromophorous compound belonging to the family of polyphenols, in a tissue of a biological entity moreover comprising a fluorescent chromophorous compound which is chlorophyll. The measurement of the polyphenols is carried out by the chlorophyll fluorescence method described in the document FR 2 830 325.

FIGS. 1 and 2 are diagrammatic representations of two embodiments of a system according to the invention. In these two embodiments, the system is presented in the form of two parts: an emitting part 10 and a receiving part 20. In the first embodiment represented in FIG. 1, the emitting 10 and receiving 20 parts are arranged on either side of the plant tissue sample 30, whereas in the second embodiment represented in FIG. 2, the emitting 10 and receiving 20 parts are arranged on the same side of a biological tissue sample 30.

Whatever the embodiment, the emitting part 10 comprises three sources of radiation 11, 12 and 13 illuminating the front surface of a sample of biological tissue 30. Each of the sources 11 to 13 is associated with a pulsed supply 14 to 16, as shown in FIGS. 1 and 2, and controlled by a synchronization signal Sₛ. The sources 11 to 13 are provided to induce the fluorescence of chlorophyll. The emitting part 10 can comprise moreover an optical filter, not shown, situated between the sources 11 to 13 and the sample 30, in order to filter the unwanted components of the radiation 11 to 13 emitted towards the sample 30.

The receiving part 20 of the system according to the invention comprises a detector 21, a silicon photodiode supplying an electrical signal as a function of the fluorescence radiations detected, associated with an optical filter F2, arranged between the sample 30 and the detector 21. The function of this filter F2 is to:
block the radiations from the excitation sources 11 to 13 from passing through towards the detector 21,
partially or totally transmit the emission of the fluorescence of the chlorophyll induced by the optical radiations emitted by the sources 11 to 13.

The receiving part 20 of the system comprises moreover a unit 22 comprising means of control and calculation making it possible on the one hand to carry out the synchronization of the supplies 14 to 16 via a synchronisation signal Sₛ and to determine the content of the first compound in the tissue sample 30 as a function of the measurement signal Sₘ provided by the detector 21.

FIGS. 3 and 4 are diagrammatic representations of the measurement principle and the optical paths of the different radiations, in the first and the second embodiment respectively. With reference to FIGS. 3 and 4, the sources 11 to 13 are preferably light-emitting diodes (LED) and illuminate the same face 31 of the sample 30. The sources 11 to 13 emit three radiations, 111, 121 and 131 respectively, intended to induce the fluorescence of the chlorophyll 33 and induce three fluorescent radiations, 112, 122 and 132 respectively. In the first embodiment the receiving part 20 of the system located on the opposite side of the leaf or the berry with respect to the emitting part 10, these are fluorescence radiations emitted by the chlorophyll towards the side opposite to the sources 11 to 13 which are detected by the detector 21. In fact, in this first embodiment, the detector 21 is provided to detect the fluorescence emitted towards the rear face 35 of the sample for measuring the content of polyphenols 34.

FIG. 4 represents the fluorescent radiations 112, 122 and 132 induced by the radiations 111, 121 and 131, and emitted by the chlorophyll towards the sources 11, 12 and 13. In this second embodiment the receiving part 20 of the system is located on the same side of the leaf or the berry as the emitting part 10, these are radiations emitted by the chlorophyll towards the sources 11 to 13 which are detected by the detector 21. In fact, in this second embodiment, the detector 21 is provided to detect the fluorescence emitted towards the front surface 31 of the leaf for measuring the content of polyphenols 34.

Whatever the embodiment, the wavelengths of each of the sources 11-13 are chosen as a function of the absorption bands of the compounds to be measured, their technical characteristics such as spectral purity or their power, their commercial availability and cost, and the commercial availability and cost of the filter or filters F2 associated with the detector 21.

The first and the second embodiment are substantially equivalent as the fluorescence is isotropic. The second embodiment is the preferred embodiment and makes it possible to use the system according to the invention in situ and remotely from the biological tissue to be characterized. Moreover, in this second embodiment the system according to the invention can be used directly on the biological entity in a non-destructive and non-intrusive fashion, i.e. without having to take a sample of a biological tissue.

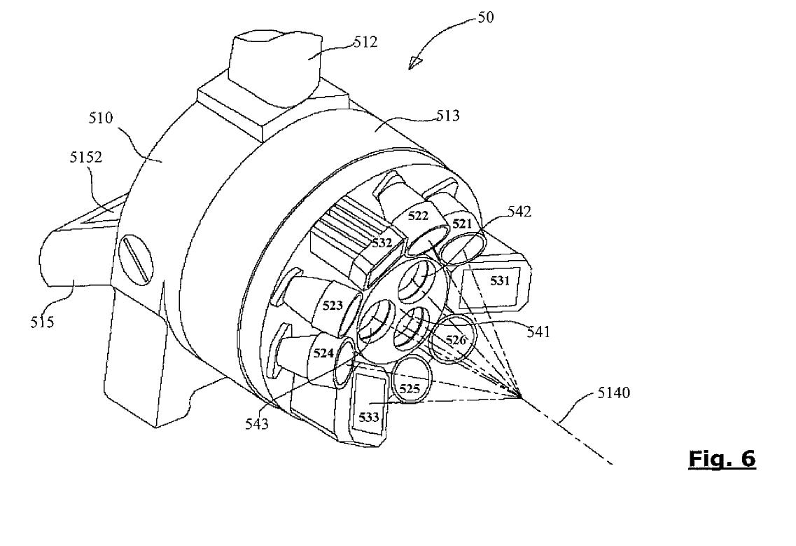

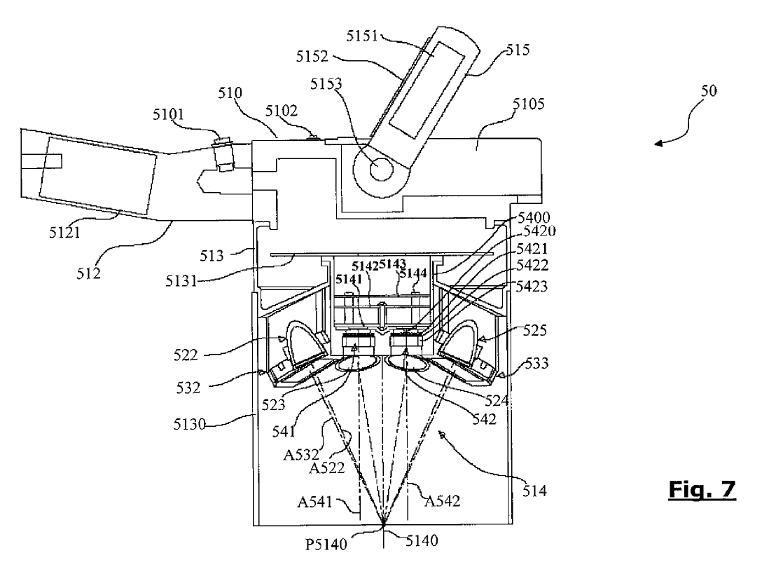

FIGS. 5 to 7 represent different views of a device 50 produced according to the second embodiment of the system according to the invention, this second embodiment being the preferred embodiment of the system according to the invention. FIG. 5 represents a front cross section, FIG. 6 represents an isometric view and FIG. 7 represents another cross section, from the side, of the device 50. The device 50 allows measurements to be made directly on a bunch 60 of grapes comprising a plurality of berries 70.

This embodiment, that will be called MULTIPLEX®, is based on a portable case 510 supplied by a battery 5121 which can be remote or integrated in the handle, provided with a measurement surface 514 and a user interface comprising a screen 5152 and control elements such as buttons or keys 5101 and 5102. This case can be held by a part forming a handle 512 containing the replaceable battery 5121 or the connector of the remote portable battery.

This case 510 also comprises a cylindrical part 513 extending towards the side opposite the interface and carrying the measurement surface at its end. The measurement surface 514 is surrounded by a shield 5130 which is more or less opaque and possibly detachable, which makes it possible to reduce interference from ambient light and to provide a pint of reference as to the optimal measurement distance relative to the measurement surface 514.

This measurement surface 514 comprises a set 540 of detectors covering the fluorescence wavelengths to be measured. In the embodiment described here, this set 540 comprises three detectors 541, 542 and 543 adjacent to each other and grouped together in an equilateral triangle in the centre of the measurement surface 514. These three detectors are oriented in directions parallel to each other about an detection axis 5140, or very slightly convergent about this detection axis 5140. Each of these detectors 541, 542 and 543 comprises a detection element, here a silicon photodiode 5420 of approximately 2 cm×2 cm, and detects the light in a determined wavelength band, blue-green, red and far red respectively. This detection band is obtained by a coloured or high-pass filter and an interferential filter. The combination of these two types of filters allows a better filtration which may be necessary, in particular to prevent the detectors receiving radiation emitted by the excitation sources.

It should be noted that the detectors directly receive the fluorescence to be measured, without using collection, convergent or collimated optics. Each detector requires only a single detection element, the photodiode 5420 (FIG. 7), chosen to be large enough to obtain a good sensitivity which makes it possible to dispense with collection optics. This detection element thus receives radiation 549 originating from all of the target zone 591 illuminated by the excitation emitters.

This arrangement makes it possible to use relatively simple detection elements, and avoid the need for collection optics. Over and above the saving on the cost of the optics, the size requirement, regulation and field depth constrains are also avoided.

The measurement surface 514 has a concave conical peripheral surface supporting several sets of emitters, which can emit excitation light in different wavelengths, distributed in a circle around the set of detectors 540.

These emitters comprise a set of ultraviolet emitters 520 comprising six UV emitters 521 to 526, distributed at 120° in three groups of two adjacent emitters, on a circle around the set of detectors 540.

Each of these sources comprises a source, here an ultraviolet LED 527, placed in a parabolic reflector 5281 forming a beam of approximately 30°. The reflectors are mounted on a base 5282 determining the position of their beam with respect to the detection axis 5140. Alternatively, the UV emitters can also use a dioptric or catadioptric device to improve the convergence of the beam emitted.

The emitters also comprise a set of visible light emitters 530, comprising three emitters 531, 532 and 533 distributed at 120° around the set 540 of detectors in the same circle as the set of UV emitters 520 and intercalated with the UV emitters. Each of these visible light emitters comprises a source comprising an array of intercalated red-, green- and blue-coloured LEDs, incorporated in a common component 534 with sides measuring approximately 4.5 cm and with a power of 3×15 W, and covered by a plate of transparent plastic forming an array of convergent micro-lenses. This common component 534 is mounted on a block 536 forming a radiator, the shape of which determines the orientation of the source with respect to the detection axis 5140. The emitter also comprises a wide bandpass coloured filter, making it possible to restrict the emissions in the wavelengths used for the fluorescence detection, in particular towards the far red.

As an alternative to the RGB (red-green-blue) sources described here, monochromatic excitation sources can also be used, for example amber-coloured sources in the form of a high-power monochrome LED array, of the order of 200 W continuously.

The excitation emitters are oriented so as to obtain a uniform illumination of the target zone 591, even when it is a heterogeneous object and/or three-dimensional.

In embodiments for short-distance applications, for example using a UV excitation, the beams of the emitters are oriented so as to converge towards the axis or the axes A541, A542 and A543 of the detectors 541, 542 and 543. More particularly, the axes A522, A532 of the excitation beams intersect with each other and with the detection axis 5140 at the same point P5140, at an optimum distance for the measurement. In the embodiment described, the convergence point P5140 is situated between 10 and 20 cm from the set 540 of detectors, for example approximately 15 cm.

The fact that the beams emitted are not collimated and exhibit a certain opening or divergence makes it possible to limit the constraints affecting the measurement distance. In fact, as the target, here a bunch of grapes 60, is situated inside the beams 529 and 539 of the emitters, it is illuminated homogeneously over its different surfaces directed towards the set of detectors 540. The fluorescence 549 emitted towards the detectors is thus sufficiently stable and homogeneous to provide true measurements of the measured zone 591.

Thus, although the measurements using the UV emitters are made at a distance of approximately 15 cm, the measurements using only the visible-light emitters can be carried out at greater distances, even up to approximately 1 m, for example for a measurement of the anthocyanins.

In other embodiments, the beams from the emitter can be oriented so as to be parallel to the detection axis, for example for applications with measurement at a significant distance.

FIG. 7 illustrates more particularly the structure of the device in this embodiment of the invention. The cylindrical part 513 of the case contains an electronic card 5131 which is annular substantially, comprising the power-supply circuits for the excitation sources, the management circuit of the excitation pulses and a circuit producing a current generator.

The detectors, and accompanying electronics are grouped together in a cylindrical detection module 5400, placed on the measurement surface 514 in the centre of the circle formed by the excitation emitters and extending in the direction of the target to be analysed. The outer surface of this cylinder carries the three detectors 541, 542 and 543.

This arrangement makes it possible to place the detectors substantially at the same level as the ends of the emitters in order for them to have a wide reception field and thus makes it possible to get close to the target.

The detection module 5400 comprises three small electronic cards 5141, 5142, 5143 essentially analogue, substantially circular, stacked along its longitudinal axis, fixed and spaced out by small columns 5144.

The first small electronic card 5141, situated on the side of the outer surface of the detection module 5400, carries the detection elements, here silicon photodiodes. For each of these detectors, the silicon photodiode 5420 receives the light to be detected through a coloured or high-pass filter 5421 and an interferential filter 5422 detachably fixed by a retaining nut 5423 in a 25.4 mm cylindrical opening, which can thus receive standard 1 inch or 25 mm filters.

Moving away from the measurement surface 514, the second small electronic card 5142 carried the circuits and amplifiers carrying out rejection of the ambient light by a negative feedback loop.

The third small electronic card 5143 carries circuits and components containing in particular track-and-hold units.

The detection module 5400 constitutes a compact assembly which can be removed from the case 510, for example for maintenance or to be replaced by a camera module or a module including one or more optical waveguides.

This detection module is connected, through the opening in the large electronic card 5131, to a processing module 5151 situated on the side of the interface and comprising all or part of the processing means: in particular an acquisition unit and calculation means.

This processing module is comprised in a part 515 of the case carrying the screen 5152, which can be inclined for good readability, and retracted into a compartment 5105 inside the case 510. This processing module can comprise a detachable connection which allows it to be exchanged easily, for example for updating or a change in function.

The electronics 5131 linked with the excitation, and 5141 to 5143 linked with the detection, and the processing module 5151 are thus arranged in different and separate electronic modules, allowing simplified maintenance. These modules are moreover separated by a certain distance, here 2 cm and for example at least 1.5 cm, which allows better dissipation of the heat generated and limits the risks of interference between the circuits that they comprise.

In the embodiment described here, the detection is synchronized with the excitation which is emitted by the excitation emitters. An excitation frequency of 1 kHz with 520 microsecond pulses has been used with success, and allows processing in real time as and when needed while covering the site. The different fluorescence measurements necessary for the establishment of the content or programmed index are interleaved within the measurement period.

Thus the management and processing means are arranged in order to:
emit a control signal for controlling the emitters by pulses,
detect the fluorescence peaks generated by these pulses, an amplification within the detectors produces the rejection of the ambient light by a negative feedback loop,
control, for example by the same control signal, the processing of the detected fluorescence peak, for example, by means of track-and-hold units; and
supply an analogue measurement of the fluorescence measurement to the acquisition unit.

In other embodiments, a synchronous detection is provided, using phase modulation between the excitation and the detection. The management and processing means are then arranged in order to:
control the emitters according to a frequency including a phase modulation,
process the fluorescence detection signal in phase demodulation and provide the fluorescence measurement.

Determination of the anthocyanin (ANTH) content of the skin of a grape berry will now be described according to the first aspect of the invention and at a variable distance.

FIG. 8 shows the absorption spectrum 82 of malvidin glycoside which is the majority anthocyanin in grapes, in 50% acidified methanol, in extinction coefficient units, the emission spectrum 83 of the chlorophyll and the transmission spectrum 84 of the filter F2 which is a Schott RG9 filter.

The source 11 is a source emitting a radiation 111 in the green (GREEN), the wavelength of which is situated in an absorption band of the anthocyanins and which induces the fluorescence of chlorophyll 33 and causes the emission of a fluorescence radiation 112. The wavelength of this source 111 can for example be around 530 nm. FIG. 8 gives the spectrum 86 of the radiation 11. An example of a GREEN source is the NS530L diode from Roithner Lasertechnik.

The source 12 emits a radiation 121 in the red (RED), where the anthocyanins absorb slightly or not at all, which induces the fluorescence of chlorophyll 33 and causes the emission of a fluorescence radiation 122, which acts as a reference for the measurement of the anthocyanin content. The wavelength of this source is preferably at 650 nm. FIG. 8 gives the spectrum 85 of the radiation 121.

Moreover the source 13 emits a radiation 131 in the blue (BLUE), where the anthocyanins absorb slightly or not at all, which induces the fluorescence of chlorophyll 33 and causes the emission of a fluorescence radiation 132, which acts as additional radiation for the measurement of the anthocyanin content. The wavelength of this source is preferably at 450 nm. FIG. 8 gives the spectrum 87 of the radiation 12.

Sources 11, 12 and 13 successively illuminate the tissue 30 by respectively emitting a radiation 111, 121 and 131. The radiation 111 is absorbed in a variable quantity by the epidermis of the skin depending on its anthocyanin content 34, while the red 121 and blue 131 radiations are not absorbed or only a little absorbed. All radiations 111, 121 and 131 induce the fluorescence of chlorophyll 33 which then respectively emits fluorescence radiations 112, 122 and 132 in the red and the near infrared, the intensities of which are proportional to the intensities of the radiations 111, 121 and 131 received by the chlorophyll 33. Measurement of the ratio of the fluorescence emissions excited by the source 11 and by the source 12 makes it possible to determine the anthocyanin content the skin of a grape berry. Moreover, measurement of the ratio of the fluorescence emissions excited by the source 12 and by the source 13 makes it possible to determine whether the reference radiation 12 is affected by the anthocyanins, the content of which increases over time. When the anthocyanin content of a grape increases, the radiation 111 is over-absorbed and begins to be saturated. Moreover, the anthocyanins also begin to affect the reference radiation. In this case, in order to carry out measurements beyond the saturation caused by too high a concentration of anthocyanins, the radiation 121, which was the reference radiation before saturation, becomes the measurement radiation after saturation and the radiation 131, which was the additional radiation before saturation, becomes the reference radiation after saturation. The radiation 111 is no longer used in the measurement. Thus, measurement of the anthocyanin content can then be carried out beyond saturation.

The anthocyanin content is then calculated according to the following expression:

ANTH_{before saturation}=logFER_ANTH_GREEN−logFER_CHL_{G}

ANTH_{after saturation}=C+logFER_ANTH_RED−logFER_CHL_{R}

with,
1. logFER_ANTH_GREEN=log(FRFred/FRFgreen)
2. logFER_ANTH_RED=log(FRFblue/FRFred) and
3. logFER_CHL_{G}=log(FRFred/FRFgreen)
4. logFER_CHL_{R}=log(FRFblueFRFred)
   In these expressions logFER_CHL_{G}=log(FRFred/FRFgreen) and logFER_CHL_{R}=log(FRFblue/FRFred), the quantities logFER_CHL_{G} and logFER_CHL_{R} being measured before the appearance of the anthocyanins in the grape skin, ANTH is the anthocyanin content of the skin of a grape berry in nanomoles/cm², FRFgreen, FRFred and FRFblue are respectively the intensities of fluorescence radiations 112, 122 and 132 induced by the radiations 111, 121 and 131 and C is a constant such that ANTH_{after saturation} is equal to ANTH_{before saturation} at the moment of saturation.

FIG. 9 shows a graph representing the development of the measured anthocyanin content in the skin a grape berry, determined according to the first aspect of the invention. The axis 91 denotes the time from which saturation compensation is carried out. The curve 92 shows the results obtained with saturation compensation according to the invention i.e. considering the reference radiation before saturation as the measurement radiation after saturation and the additional radiation before saturation as the reference radiation after saturation. Curve 93 shows the results obtained without saturation compensation. Thus, it is noted that without saturation compensation a curve 93 is obtained which decreases after saturation, while the anthocyanin content continues to increase. With compensation an increasing curve 92 is in fact obtained, demonstrating the increase in the anthocyanin content in the skin a grape berry.

Determination of the anthocyanin content in the skin a grape berry will now be described according to the second aspect of the invention and at a variable distance. According to this second aspect, the wavelength of the radiation 121 remains unchanged at 650 nm. On the other hand, the wavelength of the radiation 111 is chosen at 590 nm. This wavelength correspond to a lower absorption of the anthocyanins than the wavelength 530 nm chosen according to the first aspect of the invention and which corresponds to the anthocyanin absorption peak. FIG. 10 shows the absorption spectrum 82 of malvidin glycoside which is the anthocyanin whose content it is desired to determine, in 50% acidified methanol, in extinction coefficient units, the emission spectrum 83 of the chlorophyll and the transmission spectrum 84 of the filter F2, as well as the spectrum 85 of the radiation 121 and the spectrum 88 of the radiation 111 used in this second aspect of the invention.

The measurement principle in this second aspect of the invention is substantially similar to the measurement principle in the first aspect of the invention. Sources 11, 12 successively illuminate the tissue 30 by respectively emitting radiations 111 and 121. The radiation 111 is absorbed in a variable quantity by the epidermis of the skin according to its anthocyanin content 34, while the red radiation 121 is not. These radiations 111 and 121 induce the fluorescence of the chlorophyll 33 which then respectively emits fluorescence radiations 112 and 122 which are measured However, as the 590 nm wavelength chosen for the radiation 121 corresponds to a lower absorption than the 530 nm wavelength of the radiation 121 chosen in the first aspect of the invention described above, no saturation takes place due to the increase in the anthocyanin content of the skin of the grape berry. FIG. 11 shows a representation of:
a first curve 1111 showing the development of the measurement carried out with radiation 121 of wavelength 530 nm as a function of the anthocyanin content of the skin of the grape berry, and
a second curve 1112 showing the development of the measurement carried out with radiation 121 of wavelength 590 nm as a function of the anthocyanin content of the skin of the grape berry.
   The wavelength 590 nm corresponds to a lower absorption of the anthocyanins than the wavelength 530 nm. It is noted that for the 530 nm wavelength, curve 1111, saturation takes place very quickly, while for the 590 nm wavelength, curve 1112, there is no saturation. Moreover, in the case of the curve 1112, the results obtained are more linear than in the case of the curve 1111.

Advantageously, the first and second aspects of the invention can be combined. In fact, the additional radiation 131 described above can be used to determine whether or not the reference radiation 121 is affected by the anthocyanins. When the concentration or the anthocyanin content of anthocyanins is such that saturation takes place even with radiation 111 at a wavelength shifted with respect to the absorption peak, for example 590 nm, it is possible to consider the radiation 121, which was the reference radiation before saturation, as the measurement radiation after saturation, and the radiation 131, which was the additional radiation before saturation, as the reference radiation after saturation. FIG. 12 shows the results obtained by combining the first and the second aspects of the invention. The curve 1201 corresponds to the results of the measurement carried out with:
a radiation 111 of 530 nm,
a radiation 121 of 650 nm, and
a radiation 131 of 450 nm;
   and the curve 1202 corresponds to the results obtained with:
a radiation 111 of 590 nm;
a radiation 121 of 630 nm, and
a radiation 131 of 450 nm.
   The axis 1203 denotes the time from which saturation compensation is carried out according to the first aspect of the invention.

Moreover, the wavelengths of the radiations 111, 121 and 131 can be modified as a function of the development of the pH in the biological tissue that it is desired to characterize. FIG. 13 shows the absorption spectrum 1301 of anthocyanins at pH=1, and the absorption spectrum 1302 of anthocyanins at pH=8. It is noted that when the pH increases the absorption peak shifts towards a higher wavelength. This variation of the absorption spectrum can be taken into account in saturation compensation, whatever aspect of the invention is implemented.

A brief description will now be given of the measurement of the flavonol content (FLAV) of the skin of a grape berry, according to the third aspect of the invention. The source 11 is in this case a source emitting a radiation 111 in the ultraviolet (UV), the wavelength of which is situated in an absorption band of flavonols and which induces the fluorescence of the chlorophyll 33 and causes the emission of a fluorescence radiation 112. The wavelength of this source 11 can for example be around 375 nm. The source 12 can then emit a reference radiation 121 at 650 nm which corresponds to a wavelength that is not absorbed by the flavonols. When the measurement radiation 111 is saturated by the flavonol content in the skin then it is possible to use as the measurement radiation at 450 nm, corresponding to a lower absorption on the absorption spectrum of the flavonols. This radiation at 450 nm can be for example emitted by the source 13. The reference radiation is not modified and remains at 650 nm

The invention can also be implemented for measuring the content of other compounds which are chromophorous and slightly or not at all fluorescent, such as lycopenes in the case of tomatoes for example.

The invention is not limited to the example described in detail above and can be used for characterizing a biological tissue of any type.

1-19. (canceled)

20. Method for determining the content of a non-fluorescent chromophorous compound (34), called the first compound, in a tissue (30) of a biological entity, said biological tissue (30) comprising moreover a fluorescent chromophorous compound (33), called the second compound, said method comprising at least one iteration of the following operations:
emission by emission means (11, 12), in the direction of said tissue (30), of a first so-called measurement optical radiation (111), and a second so-called reference optical radiation (121), each chosen so as to induce a fluorescence radiation of said second compound (33), each of said first and second radiations (111, 121) being partially absorbed by said first compound (34),
measurement, by measurement means (21, 22), of said fluorescence radiations (112, 122) induced by said first and second radiations (111, 121), and
determination, from said first measurement, of the content of said first compound (34) in said tissue (30).
characterized in that said method comprises moreover at least one compensation of a measurement saturation due to too high an absorption of said measurement radiation (111) by said first compound (34), said compensation comprising a choice, for said measurement radiation, of a wavelength corresponding to a lower absorption in the absorption spectrum of the first compound (34).

21. Method according to claim 20, characterized in that it comprises moreover an emission, by the emission means (13), in the direction of said biological tissue (30), of a third so-called additional optical radiation (131), chosen so as to induce a fluorescence radiation (132) of said second compound (33), said method comprising moreover a measurement of the fluorescence radiation (132) induced by said additional radiation (131), said measurement being used for determining whether the reference radiation (121) is affected or not by the first compound (34).

22. Method according to claim 21, characterized in that, when the content of the biological tissue (30) in the first compound (34) affects the reference radiation (121), the wavelength chosen for the measurement radiation (111) after saturation corresponds to a wavelength situated around the wavelength of the reference radiation (121) before saturation.

23. Method according to claim 22, characterized in that it comprises, when the distance between the biological tissue (30) and the emission means (11, 12, 13) and the measurement means (21) is not fixed, a choice of a new wavelength for the reference radiation after saturation, said new wavelength being less affected by the content of the first compound.

24. Method according to claim 23, characterized in that when the distance between the biological tissue (30) and the emission means (11, 12, 13) and the measurement means (21, 22) is not fixed, the new wavelength chosen for the reference radiation after saturation corresponds to a wavelength which is situated around the wavelength of the additional radiation (131).

25. Method according claim 20, characterized in that, when the reference wavelength (121) is not affected by the content of the first compound (34) in the biological tissue (30), the wavelength chosen for the measurement radiation (111) corresponds to a lower absorption by the first compound (34) than a so-called limit wavelength, for which a potential saturation would be produced for an expected maximum content for the first compound (34) in the biological tissue (30).

26. Method according to claim 20, characterized in that it comprises a modification of the wavelength of the measurement radiation (111) and/or of the reference radiation (121) as a function of a physico-chemical characteristic having an influence on the absorption spectrum of the first compound (34) and/or on the fluorescence spectrum of the second compound (33).

27. Method according to claim 20, characterized in that at least one of the reference radiations (121) and the measurement radiations (111) is emitted at a predetermined intensity and the other radiation is emitted at a variable intensity so that the fluorescence radiations induced (122, 112) by each of said reference radiations (121) and measurement radiations (111) are of equal intensity.

28. Method according to claim 27, characterized in that the intensity of the measurement radiation is adjusted according to a so-called control signal, relating to the fluorescence radiations induced, on the one hand by the measurement radiation and on the other hand by the reference radiation.

29. Method according to claim 20, characterized in that the intensities of the measurement radiations (111) and the reference radiations (121) vary alternately, by phase shifting, at a predetermined frequency.

30. Method according to claim 20, characterized in that it comprises a synchronization of the radiations (111, 121, 131) by phase modulation.

31. Method according to claim 20, characterized in that each of the measurement radiations (111) and reference radiations (121) is emitted in the form of pulses.

32. Method according to claim 20, characterized in that the determination of the content of the first compound (34) comprises a calculation of the ratio:
$$\frac{fluorescence induced by the reference radiation ( 121 )}{\begin{matrix}fluorescence induced by \\ the measurement radiation ( 111 )\end{matrix}} .$$

33. Method according to claim 20, characterized in that it comprises a determination of the development, over time, of the content of the first compound (34) in the biological tissue (30).

34. Method according to claim 23, characterized in that the biological tissue is the skin of a grape berry and the second compound is chlorophyll:
when the first compound is an anthocyanin:
the wavelength of the measurement radiation before saturation is between 500 and 600 nm,
the wavelength of the reference radiation before saturation is between 600 and 700 nm,
the wavelength of the additional radiation is between 400 and 500 nm,
the wavelength of the measurement radiation after saturation is between 600 and 700 nm,
the wavelength of the reference radiation after saturation is between 400 and 500 nm; and
when the first compound is a flavonol:
the wavelength of the measurement radiation before saturation is between 300 and 400 nm,
the wavelength of the reference radiation before saturation is between 600 and 700 nm,
the wavelength of the measurement radiation after saturation is between 400 and 500 nm,
the wavelength of the reference radiation after saturation is between 600 and 700 nm.
